(19) United States
(12) Patent Application Publication
(10) Pub. No.: US 2008/0154372 A1
(43) Pub. Date: Jun. 26, 2008

(54) OSTEOCHONDRAL IMPLANT USING A GROWTH FACTOR CONCENTRATION GRADIENT FOR REPAIR OF BONE AND CARTILAGE TISSUE

(76) Inventor: Steven M. Peckham, Memphis, TN (US)

Correspondence Address: FOX ROTHSCHILD, LLP, 997 LENOX DRIVE, LAWRENCEVILLE, NJ, 08648, US

(21) Appl. No.: 11/614,665

(22) Filed: Dec. 21, 2006

(51) Int. Cl. A61F 2/28

(52) U.S. Cl. 623/16.11; 623/11.11

(57) ABSTRACT

An osteochondral device is provided comprising an implant having a growth factor concentration gradient. The implant can be porous with a higher concentration of growth factors dispersed in the portion of the implant where new bone tissue is needed and a lower concentration of growth factors dispersed in the portion of the implant where new cartilage tissue is needed.

### FIELD OF INVENTION

The present invention generally relates to a medical implant and methods for promoting new bone and cartilage growth using a growth factor concentration gradient.

### BACKGROUND OF THE INVENTION

Trauma or frequent strain on articular joints can cause lesions to articular (hyaline) cartilage and fractures to the sub-chondral bone. If the injury is not treated, it can progress into degenerative diseases, for example osteoarthritis, osteoporosis, Paget's disease, or osteohalisteresis. These lesions, often refereed to as osteochondral defects, are difficult to remedy. Treatment of damaged cartilage is hindered by finding suitable implant materials and by hyaline cartilage's low reparative capabilities.

Whereas damaged sub-chondral bone is successfully healed by osteoclasts and osteblasts, cells that resorb and deposit bone minerals, hyaline cartilage lesions generally do not form new tissue. Hyaline cartilage defects heal in a manner where the repaired tissue lacks the structural and physical properties of healthy cartilage (fibrocartilage) and will degenerate over time. This is due to the small population of type II collagen forming chondrocytes cells in hyaline cartilage and that hyaline cartilage is avascular, lacking nerves, blood vessels and lymphatic systems, which limits the healing and repair of cartilage defects.

For osteogenic and chondrogenic tissue repair, certain classes of proteins, for example bone morophogenetic proteins (BMPs), a non limiting example being BMP-2, stimulate growth of both new bone and new cartilage tissue. Implants containing growth factors allow attachment, proliferation, and differentiation of migratory progenitor cells involving bone forming osetoblasts and cartilage forming chondrocytes.

The prior art disclose devices or gels to treat and repair damaged cartilage. U.S. Pat. Nos. 6,852,125, 6,632,246, and 6,626,945 disclose artificial cartilage repair plugs used individually or in combination with other plugs. The plugs are inserted into voids left by the removal of diseased cartilage by the surgeon. They are made from a biocompatible artificial material, have varying layered and bridged configurations, and can have a plurality of anchoring elements. Certain embodiments have the plugs as anchors for a flowable polymer used to fill a void in the cartilage defect and the sub-chondral bone.

U.S. Pat. No. 7,067,123 discloses a gel for cartilage repair. The gel is a mixture of milled allograft cartilage, a bio-absorbable material, and optional additives. The gel is placed in a lesion or defect that has been removed by boring and then it is fixed in place with a periosteal cap.

U.S. Pat. No. 6,743,232 discloses a device that is anchored into the sub-chondral bone for cartilage repair. The device has a platform for holding a tissue sample, for example an allograft of cartilage. A post extends from the platform and anchors the platform into bone tissue by ribs with sharp edges that are attached to the post.

U.S. Pat. No. 6,582,471 discloses a device for cartilage repair having a porous bio-degradable implant associated with a composition for in vivo cartilage repair, wherein the device is placed in a cartilage defect. The composition is a mixture derived from bone, cartilage, tendon, meniscus or ligament or a synthetic mimic of such a mixture encapsulated in nano-spheres.

U.S. Pat. No. 7,041,641 discloses a cartilage repair plug that involves admixing growth factors of constant concentration in various matrices to enhance cartilage repair.

U.S. Pat. No. 6,575,986 discloses a scaffold fixation device for use in articular cartilage repair. The device has a platform with a post that extends from the platform and is inserted into a hole formed in the bone. The post has various configurations of ribs that extend from the side surfaces of the post. The device fastens an articular cartilage scaffold to underlying bone tissue.

U.S. Pat. No. 6,514,514 discloses a device and method for regeneration and repair of cartilage lesions. The device is a cartilage repair implant in the shape of a sheet. The device can be cut or shaped to fit cartilage tears of various shapes and sizes and to cover the entire surface of the damaged tissue. The repair implant is associated with cartilage inducing compositions made of various chondrogensis-enhancing proteins.

U.S. Pat. No. 5,632,745 discloses a method for surgically implanting a bio-absorbable cartilage repair system into a cartilage defect.

U.S. Pat. No. 6,371,958 provides for a scaffold fixation device, which fastens an articular cartilage scaffold to underlying bone.

U.S. Pat. No. 6,468,314 discloses a bio-absorbable cartilage repair system that allows for vascular invasion and cellular migration between the system and the healthy area of articular cartilage and bone.

U.S. Pat. No. 7,041,641 discloses a cartilage repair plug that involves admixing growth factors of constant concentration in various matrices to enhance cartilage repair. However, osteochondral implants having a implant with a constant BMP concentration can induce bone formation under conditions where true bone formation would, and should, not occur, such as newly formed hyaline cartilage tissue in direct contact with the implanted device.

Osteochondral defects may be treated with implants in mosaicplasty procedures. This technique involves boring holes in the base of the damaged cartilage and the underlying subchondral bone. The holes are then filled with autologus cylindrical plugs made from bone and cartilage tissues in a mosaic fashion. This procedure can be compromised if the transplanted tissue is diseased, if there is damage to the collagen forming chondrocytes, or a wearing of the graft over time.

Another procedure for treating osteochondral defects involves transplanting large allografts of bone and articular cartilage to the damaged joint. A drawback to this procedure is that there must be a fresh donor, the tissue must be stored at low temperatures and used within a month to ensure a greater than 50% cell viability.

Many therapeutic methods are available to repair bone and cartilage defects in isolation, examples being arthoscopic debridement, lavage, repair stimulation treatments, and the use of auto, allograft, or synthetic cartilage plugs. However these techniques do little to promote the simultaneous growth of new articular cartilage and new bone tissue to repair osteochondral defects.

Arthroscopic debridement and lavage removes degenerative cartilage debris from the cartilage device by irrigating the joint with salt and lactate solutions. These methods provide temporary relief of pain but do little for the formation of new cartilage tissue.

Microfracture procedures involve the puncturing of small holes into the subchondral bone to induce bleeding. A blood clot is formed when blood and bone marrow seep onto the damaged cartilage, which releases cartilage building stem cells. Like arthroscopic debridement and lavage, microfracture procedures produce cartilage tissue that is fibrous in nature and degenerates over time. Grafting procedures involve transplanting allografts or autografts of cylindrical implants having a bone base with an articular cartilage cap. These plugs fill holes bored into the subchondral bone and help stimulate the repair of cartilage defects.

Accordingly, a need exists for an osteochondral implant that effectively promotes growth of new bone and cartilage tissue without intergrowth between the two when repairing osteochondral defects.

### SUMMARY OF THE INVENTION

The present invention overcomes the drawbacks of the prior art by providing a novel osteochondral implant that comprises a growth factor concentration gradient. The implant is inserted into a surgically prepared defect that extends from the surfaces of hyaline cartilage into sub-chondral bone tissue and promotes growth of both new bone and cartilage tissue.

It is an object of the invention wherein a higher concentration of growth factors is associated with the area of the implant in contact with sub-chondral bone tissue than the area of the implant that is not.

It is an object of the invention wherein a lower concentration of growth factors is associated with the area of the implant in contact with cartilage tissue than the area of the implant that is not.

It is an object of the invention wherein the implant is made from materials selected from the group comprising ceramic material, a natural polymer such as collagen or chitosan, a biodegradable synthetic, or a non-biodegradable synthetic polymer or any combinations thereof.

It is another object of the invention wherein the implant can be fashioned into many shapes and sizes, such non-limiting examples of shapes being cylindrical, oval, ovoid, elliptical and the like.

Another object of the invention generally includes the implant being associated with bioactive agents selected from a group consisting of cells, pain-reducing agents, anti-inflammatories, antibiotics, and any combination thereof.

It is yet another object of the invention wherein the implant is porous.

It is an object of the invention wherein the implant is non-porous.

It is an object of the invention wherein the implant has a porous exterior and non-porous interior.

It is an object of the invention wherein the implant has a porous exterior and porous interior.

It is an object of the invention wherein the implant is a hollow cylindrical plug.

It is an object of the invention wherein the implant is made from dissimilar materials.

It is another object of the invention wherein the pore diameter is greater on the implant surface in contact with sub-chondral bone than that which is not.

It also within the scope of the invention to detail a method of creating an osteochondral implant having a body comprising a porous or non-porous implant with a growth factor concentration gradient disposed in or about the implant.

### BRIEF DESCRIPTION OF FIGURES

FIGS. 1-16 respectively illustrate first through sixteenth embodiment implants.

### DETAILED DESCRIPTION OF THE INVENTION

Definitions

“Growth factors,” “Bone Morphogenetic Proteins,” or “BMPs” generally refer to a class of proteins that induces the growth of new bone, such as endochondral bone, or new cartilage, such as hyaline cartilage, by recruitment of cells, cell proliferation and cell differentiation. An example of a non-limiting selection of BMPs includes BMP-1, BMP-2, rhBMP-2, BMP-3, BMP-4, rhBMP-4, BMP-5, BMP-6, rhBMP-6, BMP-7 (OP-1), rhBMP-7, BMP-8, BMP-9, BMP-10, BMP-11, BMP-12, BMP-13, BMP-14, BMP-15, BMP-16, BMP-17, BMP-18; Growth and Differentiation Factors (GDFs), such as GDF-5 and rhGDF-5; Cartilage Derived Morphogenetic Proteins; LIM mineralization proteins; platelet derived growth factor (PDGF); transforming growth factor β (TGF-β); insulin-related growth factor-I (IGF-I); insulin-related growth factor-II (IGF-II); fibroblast growth factor (FGF), and beta-2-microglobulin (BDGF II).

“Porosity,” “porous,” or “porous implant” generally indicates a body of the osteochondral implant having a molecular arrangement that is permeable, allowing for the passing of liquids, bone or cartilage tissue, growth factors or cells into the interior and exterior surfaces of the implant.

“Implant” or “osteochondral implant” generally refers to a device capable of having a growth factor and other bioactive agent concentration gradient associated with its surfaces prior to implantation within a patient. The implant can be of various shapes and sizes.

“New bone” generally refers to the growth of new bone tissue promoted by growth factors associated with the implant. New bone forms when mesenchymal cells differentiate under the influence of growth factors, such as BMPs, thereby supplying bone forming osetoblast cells.

“New cartilage” generally refers to the growth of new cartilage tissue, such as hyaline cartilage, promoted by growth factors associated with the implant. New cartilage forms when chondrocytes, embedded in a collection of collagen, proteoglycans, proteins, and water, form the necessary collagen to create connective tissue.

Various embodiments of the invention are further detailed herein. Although the present invention is primarily intended to treat and repair osteo-chondral defects, there are no intentions for the use of these words to limit the scope of the invention. Additionally, devices as described in the following contemplate their respective structures as they would exist prior to implantation within a patient. Ideally, the device is shipped, with the structure as described in the following, to a medical practitioner in a suitably sealed and sterilized kit. Packaging of devices in kits is known in the art, and any such suitable packaging method may be employed to provide a kit comprising an embodiment implantable device.

Any and all use of specific language and references are for detailing different embodiments of the same. In addition, and despite explicit reference to only the following embodiments, any and all alterations and further modifications of the invention, as would occur to one having ordinary skill in the art, are intended to be within the scope of the invention.

### Implant—General Embodiment

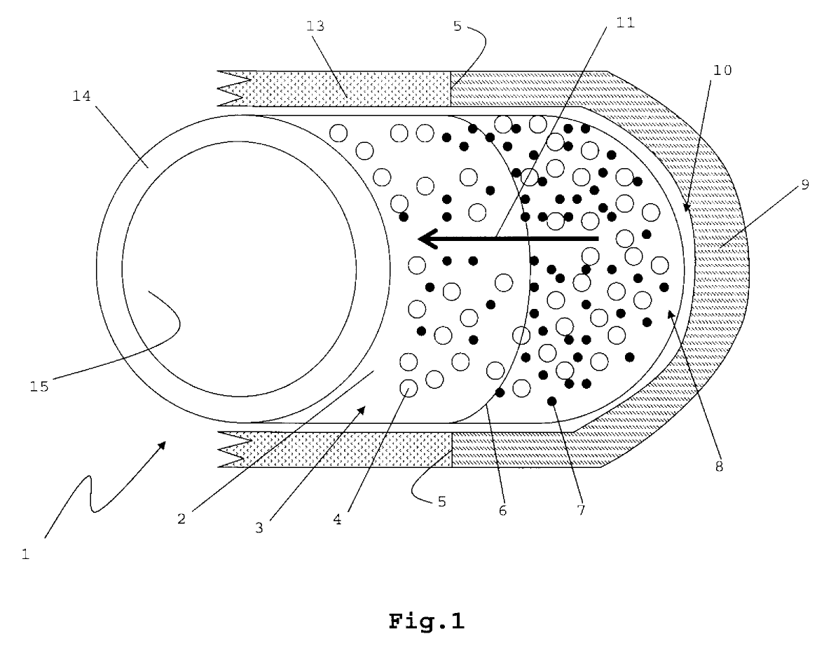

FIG. 1

FIG. 1 shows a hollow cylindrical implant 1. The implant 1 may have a uniform size, with circular end surfaces that are connected by a single continuous cylindrical surface between the end surfaces. The cylindrical implant 1 may have a ring-like cross-section, with an external diameter from about 1 cm to about 1.5 cm, and an internal diameter that is from 40% to about 80% of the external diameter.

The implant 1 has an external surface 2 comprising a plurality of pores 4 and a therapeutically effective amount of a growth factor 7. An internal surface 15 of the implant 1 may be substantially non-porous, and may or may not have growth factors 7. The pores 4 may be, for example, from 50 microns to 1000 microns in size, and may or may not be substantially evenly disposed across the external surface 2.

One of ordinary skill in the art should appreciate that the implant 1 may be molded into various shapes and sizes, for example plug, to accommodate a wide range of osteochondral defects; it may be desirable, however, to ensure that the implant retains similar physical, mechanical, and load bearing properties of the surrounding tissue, such as the bone 9 and cartilage 13.

Suitable Materials for Making the Implant

Materials may be selected based on demonstrated ability to bind or deliver growth factors and to support cell attachment and tissue ingrowth. In certain embodiments, materials may mixed together during manufacturing, such as creating a collagen calcium phosphate slurry and then freeze drying the slurry. In alternative embodiments, the implant may be made from two, or possibly more, individual sections; these sections may be created and bound together with polymer, or may be glued together. In other embodiments, a polymer coating on the exterior of the implant may hold internal sections in a desired configuration. The implant 1 may therefore be made from various materials, for example but not limited to, synthetic bio-degradable polymers, synthetic non-biodegradable polymers, natural polymers, ceramics and any combinations thereof.

Suitable non-limiting examples of synthetic biodegradable polymers include a-hydroxy acids, such as poly-lactic acid, polyglycolic acid, enantioners thereof, co-polymers thereof, polyorthoesters, and combinations thereof.

Suitable non-limiting examples of synthetic non-biodegradable polymers include hydrogels such as PVA, delrin, polyurethane, polyethylene, co-polymers thereof and any combinations thereof.

The implant 1 may be made from natural polymers including, without limitations, collagen, elastin, silk, hyaluronic acid, chytosan, and any combinations thereof.

Methods for producing polymers are described, for example, in U.S. Pat. No. 5,290,494 (Coombes) incorporated herein by reference in its entirety. Generally, these methods involve the steps of: (1) polymer dissolution in a solvent; (2) casting the solution in a mold; (3) gel formation in situ; (4) removal of the shaped gel from the mold; and (5) drying to obtain solid material in relatively thick sections.

Since at least some of these polymers are generally hydrophobic, it may be advantageous to add compounds which increase the hydrophilic properties of these polymers and thus promote entrance of intercellular fluids into the pores of those polymers.

Suitable compounds include, without limitation, surfactants. Preferably, the surfactants are physiological surfactants, including, without limitation, non-toxic anionic, cationic, amphoteric or nonionic surfactants compatible with the growth factors 7 and materials of the implant 1.

Specific examples of such surfactants include, without limitation, metal soaps of fatty acids, alkyl aryl sulfonic acids, linear aklylbenzene sulfonates, alky sulfates, alcohol ethoxylates, alcohol ethoxy sulfates, alkylphenol ethoxylates, alpha olefin sulfonates, secondary alkane sulfonates, and alpha olefin sulfonates, as disclosed in U.S. Pat. No. 5,935,594 (Ringeisen), incorporated herein by reference in its entirety.

Implant materials are not limited to merely natural or synthetic polymers. Multiple sources may serve as suitable materials for the implant. In one embodiment, the implant may be formed from ceramic materials. These materials may include porous calcium phosphate, such as, for example, hydroxyapatite (HA), tri-calcium phosphate (TCP) or any combination thereof, including, without limitations, approximately 30% HA and approximately 70% TCP. As per the listed implant materials, a calcium phosphate insert inherently binds BMPs to facilitate bone formation that synthetic polymers may not. It also has sufficient residence time in the patient to allow new bone to form before it is degraded by the body that synthetic polymers often don't.

Several processes have been developed for synthesizing bioceramic parts incorporating more or less controlled macroporous architecture. Macroporous ceramics are generally obtained by adding porogenic agents, such as naphtalene or camphor particles, polymer microbeads of polyethylene, polymethyl metacrylate, PVB and the like, during the shaping step of the ceramic part by slip casting or dry pressing.

The porogenic particles are sublimated or thermally decomposed before the final thermal densification treatment, thus leaving their mark in the form of pores in the final ceramic product. Other techniques, such as those reported in European patent application EP-A-253506, or in international patent application WO 98/38949, are also applicable.

For example, pore size in calcium phosphate materials may be determined by creating a polymer skeleton, pouring in a ceramic slurry, and removing the polymer by heating. For a hollow implant, pores may be incorporated into a molding process or added after molding, such as using a laser to create holes through the walls of the implant. Pore size may be, for example, from 100 microns to 400 microns.

Another possible way for obtaining calcium phosphate bodies with interconnected macroporosity is to exchange the carbonate ions of a coral block against orthophosphate ions in aqueous solutions of phosphates under high temperature and pressure; the so-obtained ceramic parts have the crystallographic structure of HA and the porous structure of the parent coral.

Growth Factor Concentration Gradient

The growth factors are disposed over, or within, the implant so as to create a concentration gradient of growth factors from a first region to a second region of the implant. For example, as shown in FIG. 1, the implant 1 may conceptually be divided into two regions, 3 and 8, along a concentration dividing line 6. The concentration dividing line 6 may or may not evenly bisect the external surface 2 of the implant 1. In certain preferred embodiments, the concentration gradient runs across a monolithic device, i.e. an implant that is made from a single uniform piece.

Within the first region 3, a relatively low concentration of growth factors 7 is present. Alternatively, there could be a lack of growth factors 7 in the first region 3 as well. Within the second region 8, a relatively higher concentration of growth factors 7 is present. The concentration gradient may initially form substantially a step-like function along the concentration dividing line 6. For example, the concentration of growth factor 7 in the first region 3 may be 0.05 to 0.5 mg/mL, and in the second region 8 may be 0.5 to 4 mg/mL.

The concentration gradient may, for example, vary within a limited range, such as a few millimeters, around the concentration dividing line 6, and then reach its global maximum and minimum values outside of this range. The arrow 11 indicates the net flow of the growth factor 7 across the surface of the implant 1 from the second region 8 to the first region 3.

As shown in FIG. 1, the implant 1 may be inserted into a surgically prepared defect 10 that extends from the surfaces of hyaline cartilage 13 into sub-chondral bone 9. When inserted into the defect 10, the implant 1 may ideally be positioned so that the concentration dividing line 6 substantially aligns with an intersection point 12 of the two tissue types 9 and 13.

Hence, the first region 3 may be substantially embedded within first tissue type 13, while second region 8 is substantially embedded with second tissue type 9. Alternatively, depending upon how the two tissue types 9 and 13 react to different concentrations of the growth factor 7, it may be desirable that the concentration dividing line 6 be offset, perhaps by as much as a few millimeters, within one or the other regions of tissue type 9, 13.

For example, as shown in FIG. 1, the second region 8 of the implant 1 may have a relatively high concentration of a BMP 7, such as rhBMP-2, and the first region 3 may have a relatively low concentration of the rhBMP-2 7. As indicated earlier, the BMP may have a concentration of 0.05 and 0.5 mg/mL in the first region 3, and have a concentration of 0.5 to 4 mg/mL in the second region 8. It will be appreciated, however, that concentrations may be affected by differing delivery methods and release kinetics. Additionally, different growth factors 7 may have different concentration ranges.

The second region 8 may be embedded within bone 9, and the first region 3 may be embedded within cartilage. The concentration dividing line 6 may align with the intersection point 5 of the bone 9 and the cartilage 13. However, it may be desirable to shift the alignment of the concentration dividing line 6 with respect to the tissue intersection line 5. For example, relatively high concentrations of BMP may cause the formation of bone in cartilage, whereas low concentrations of BMP tend not to lead to the formation of cartilage in bone. Hence, to further avoid the risk of new bone forming in the cartilage region 13, it may be desirable to have the low concentration region 3 overlap slightly into the bone region 6.

Various methods may be employed to create an initial concentration gradient within the implant 1 to form the first region 3 of relatively low concentration of the growth factor 7 and the second region 8 with a relatively high concentration of the growth factor 7. For example, the material used for the implant 1 may be porous and may bind to the growth factor 7; for example, calcium-phosphate based ceramics, such as HA, tri-calcium phosphate or the like, naturally tend to bind to BMP. The body of such an implant 1 may be used somewhat like a filter during the manufacturing process. A growth factor solution, such as a BMP solution, may be added to one end of the implant 1 that is to correspond to the second region 8. As the growth factor fluid flows through the carrier 1, the growth factor 7 binds to the carrier 1. A concentration gradient is thus naturally formed, as the concentration of the growth factor 7 is lower farther from where the growth factor solution is added, i.e., is lower in the first region 3. Such a processing step will thus create an implant 1 with a smoothly sloping growth factor concentration gradient along the longitudinal axis of the implant 1, rather than a step-like concentration gradient along this axis. Note that, depending upon how the growth factor solution is added to the implant 1 during the manufacturing step or during surgery, a growth factor concentration gradient may or may not further be created along the radial direction (perpendicular to the longitudinal direction) of the implant 1.

In an alternative method, the binding of the growth factors 7 to the implant 1 is regulated by controlling the distribution of the materials in the implant 1. For example, materials having a high affinity with the growth factor 7 may be used for the second region 8, while materials with a low affinity for the growth factor 7 may be used for the first region 3. When a solution containing the growth factor 7 is added to the implant 1 (for example, the implant 1 is immersed in, or sprayed with, such a solution), more of the growth factor 7 will bind in the second region 8 rather than in the first region 3. As indicated earlier, calcium phosphate ceramic granules have a relatively high affinity for BMP, and so in one embodiment the second region 8 may be formed from, or otherwise comprise, such granules.

Alternatively, the implant 1 may be formed from two or more separate sections that are later bound together, each having its respective concentration of the growth factor 7. For example, the first region 3 could be made independently as a section and provided a desired concentration of the growth factor 7. Similarly, the second region 8 could be made independently of the first region 3 and provided another, higher, concentration of the growth factor 7. Then, the two sections 3,8 may be glued, bonded, mechanically or otherwise joined together to form the implant 1. Such an implant 1 would have a substantially step-like concentration gradient. Affixing the two sections 3,8 together may be performed during the manufacturing step, or may be performed by the medical practitioner just prior to implantation into the patient.

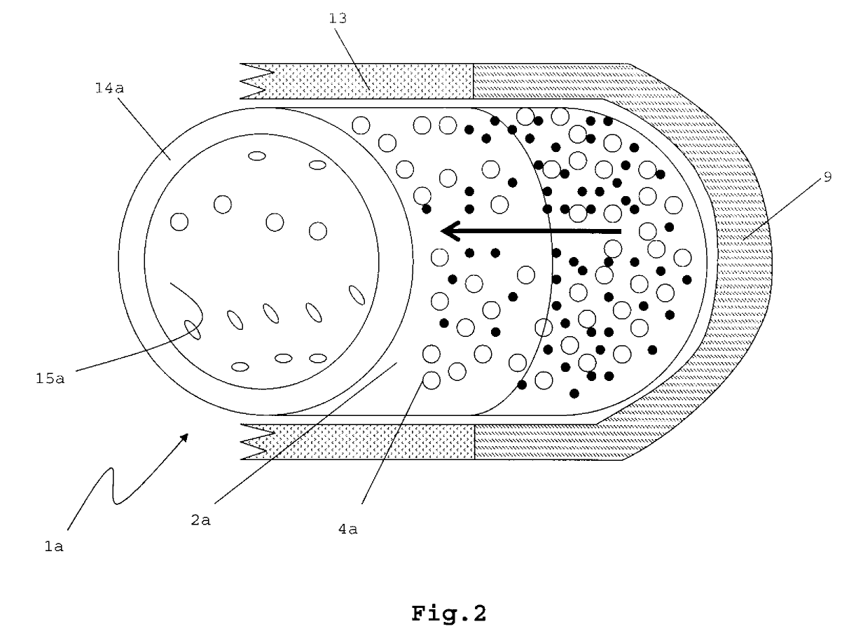

### Various Implant Embodiments—FIGS. 2-16

FIG. 2

As indicated in FIG. 1, the implant may be treated to provide a porous external surface 2. Alternatively, as shown in FIG. 2, an implant 1a may be formed from a device that is similar to the previous embodiment 1, but which is porous throughout its volume. The implant 1a is thus provided pores 4a on both the external surface 2a and internal surface 15a. Although not completely shown, the internal volume 14a of the implant 1 is porous.

Creating a porous structure in or about the various embodiments of the implant 1, 1a-1o may enhance the ability of cell attachment, and allow for cellular migration and overgrowth of tissue layers. A desired characteristic of porosity is the ingrowth of bone tissue 9 into the surfaces of the implant 1, 1a-1o, which helps to transfer load from the implant 1, 1a-o to the newly formed surrounding bone tissue. The pores 4, 4a-4o may have sizes substantially equal to those of the first embodiment 1.

As previously indicated, a porous implant 1 may be achieved by many methods. Crystals or powders, for example but not limited to, sucrose, salt, calcium carbonate or sodium bicarbonate may be added during the molding process of a implant 1, 1a-1o made from a synthetic polymer. The crystal or powder additive will embed into the implant and upon drying or dissolution of the additive, leave the implant 1, 1a-1o in a porous state.

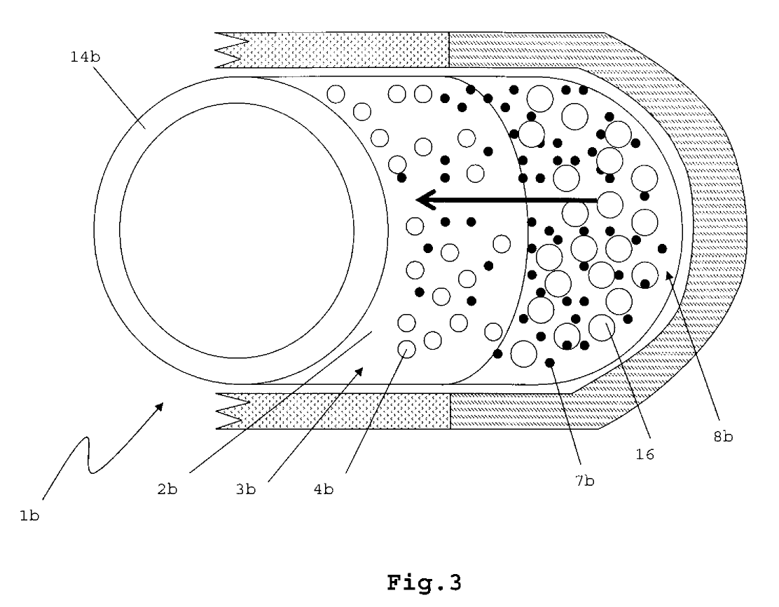

FIG. 3

Pore size of the implant 1, 1a-1o may be controlled by the type and amount of porgen used. FIG. 3 provides for an osteochondral implant 1b with various pore sizes 4b, 16 in different sections of the implant 3b, 8b to accommodate the diffusion mechanics associated with a growth factor concentration gradient and to provide better surfaces for tissue ingrowth. The implant 1b may have an external surface 2b having a first region 3b with a relatively low concentration of a growth factor 7b, and a second region 8b with a relatively high concentration of the growth factor 7b.

Within the first region 3b, pores 4b may be relatively small, for example with an average size from 50 to 200 micron. Within the second region 8b, pores 16 may be relatively large, with average sizes from 400 to 1000 microns. The porous implant 14b may be provided, for example, by a surface treatment that exposes the external surface 2b of the implant 1b to a plasma, such as a hydrogen peroxide plasma, or by milling, sand blasting or the like. Without wishing to be bound by theory, it is believed that by controlling the size of the pores 4b and 16 within the regions 3b and 8b, it is possible to control the rate of diffusion of the growth factor 7b between the regions 3b and 8b. Alternatively, the second region 8b may be more porous than the first region 3b.

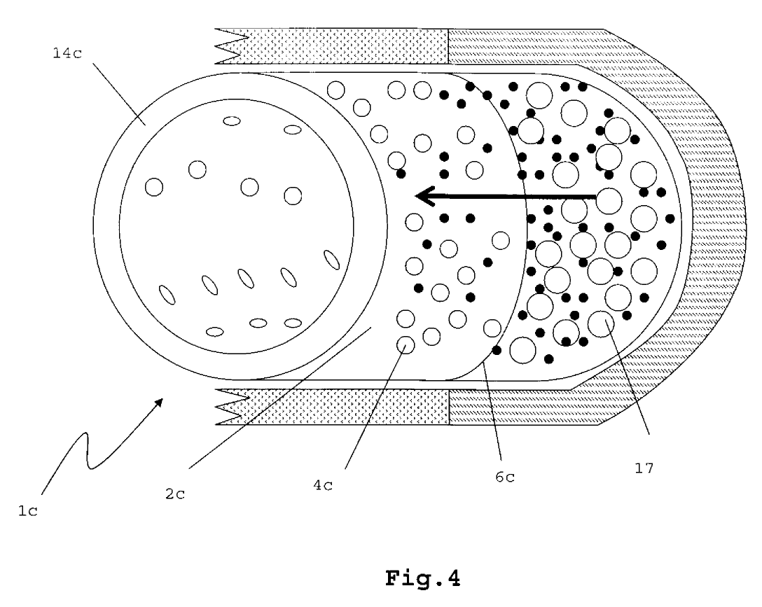

FIG. 4

As shown in FIG. 4, an implant 1c has pores 4c, 17 that are dispersed throughout the volume 14c of the implant 1c, analogous to the second embodiment implant 1a, but the sizes of the pores 4c, 17 are controlled as in the third embodiment 1b.

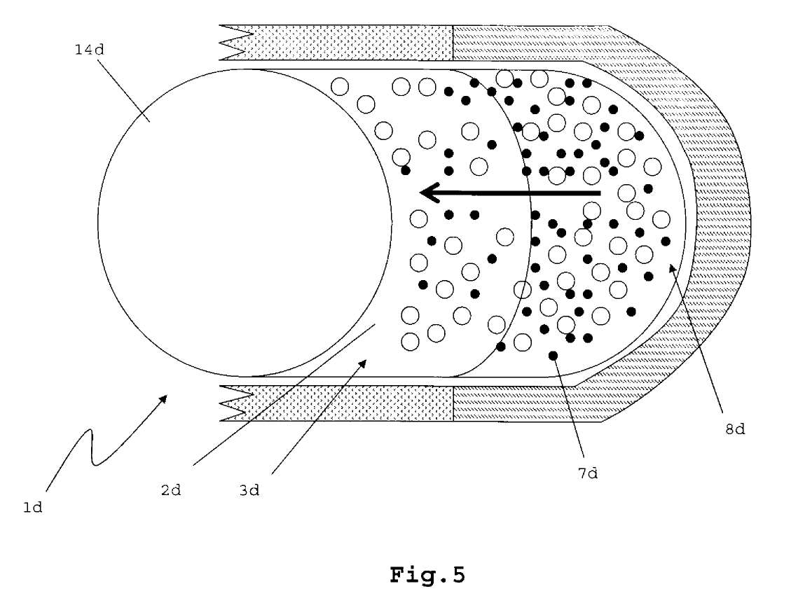

FIG. 5

It is not necessary, of course, for the implant to have a hollow core. On the contrary, it is quite possible for the implant to be solid throughout, which may or may not be porous within its volume. For example, as shown in FIG. 5, an implant 1d may have a solid inner surface 14d and a porous outer surface 2d. The implant 1d has a relatively low concentration of a growth factor 7d in a first region 3d, and a relatively high concentration of the growth factor 7d in a second region 8d. The fifth embodiment implant 1d is analogous to the first embodiment 1, but the implant 1d is solid, rather than hollow, throughout its cross-section.

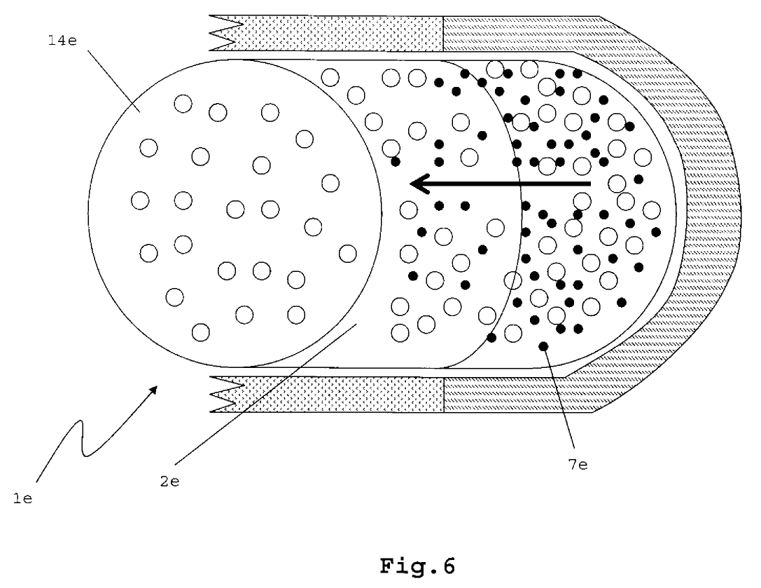

FIG. 6

Implants with solid bodies may have variations similar to those indicated in the prior embodiments. For example, as shown in FIG. 6, an implant 1e has a solid porous body, rather than just having a porous external surface 2e. The growth factor 7e may have a concentration gradient across the external surface 2e, and may also have a concentration gradient through the volume 14e of the implant 1e. For example, the concentration of the growth factor 7e may be substantially constant along the radial axis of the implant 1e, but may change as a function of the longitudinal axis.

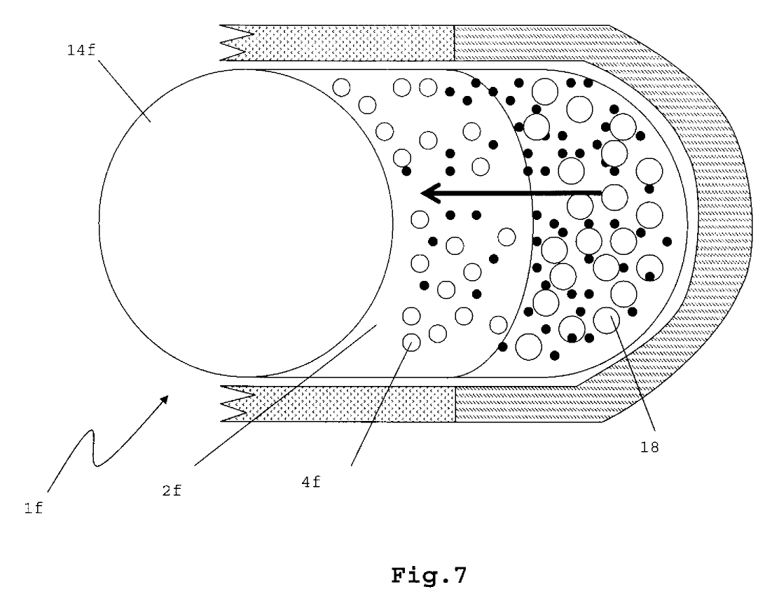

FIG. 7

As shown in FIG. 7, an implant 1f may have a solid interior with a porous external surface 2f. The size of the pores 4f and 18 may be controlled as in the third embodiment implant 1b.

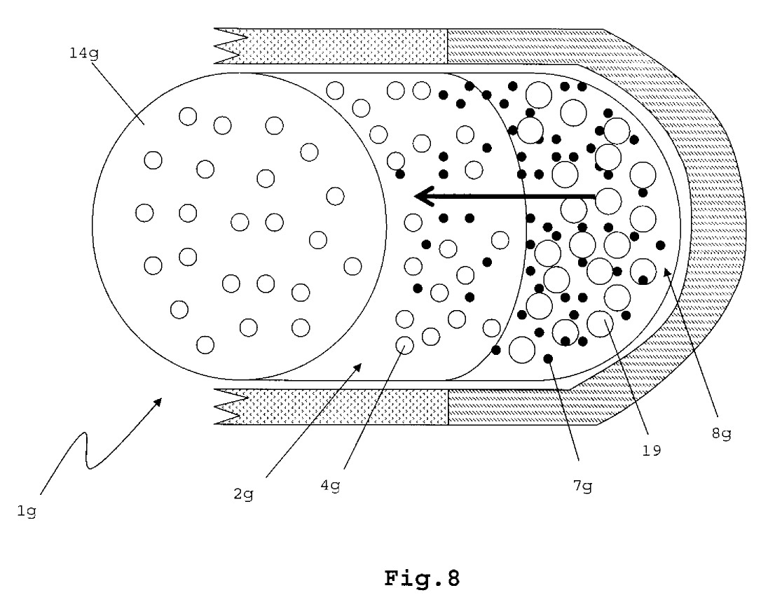

FIG. 8

FIG. 8 shows an implant 1g having a solid porous interior. The pores 4g, 19 are present throughout the volume 14g of the implant 1b. As in the previous embodiment 1f, the size of the pores 4g, 19 may vary upon the concentration region 2g, 8g, wherein larger pores 19 may be, for example, situated in the second concentration region 8g.

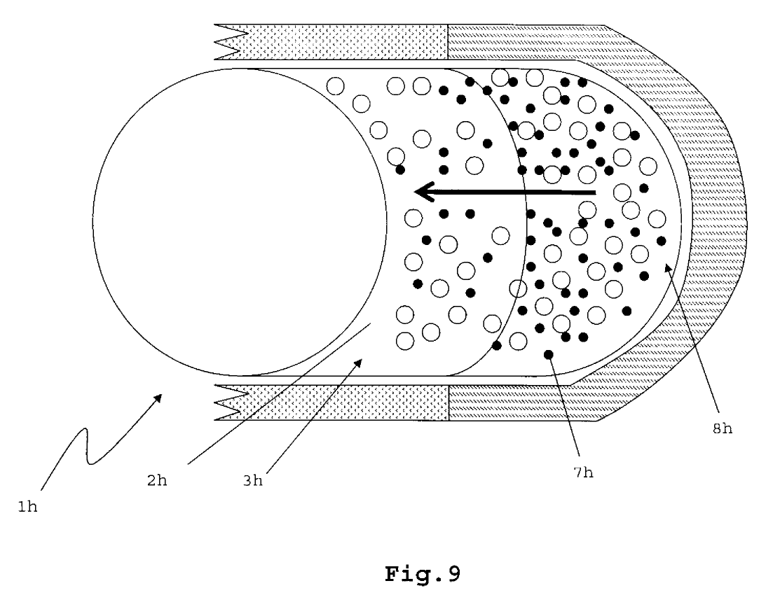

FIG. 9

It is not necessary that the implant be made entirely of the same material. It may be desirable to provide the implant with a compound structure, in which one region or section of the implant is made from a first material type, and a second region or section of the implant is made from a second material type. For example, the embodiment shown in FIG. 9 includes a solid implant 1h having a first region 3h made from a material that is relatively quickly absorbed by the body, and a second region 8h made from a material that is absorbed more slowly by the body.

The first region 3h may be made, for example, from collagen, and the second region may be made, for example, from a biocompatible ceramic. A relatively high concentration of a bioactive agent 7h may be present in the second region, whereas a relatively lower concentration of the bioactive agent 7h may be present in the first region 3h. The external surface of the solid implant 2h may be porous. Any suitable means may be employed to bind the first region 3h to the second region 8h, such as mechanical interlocking, welding, pressure or adhesives.

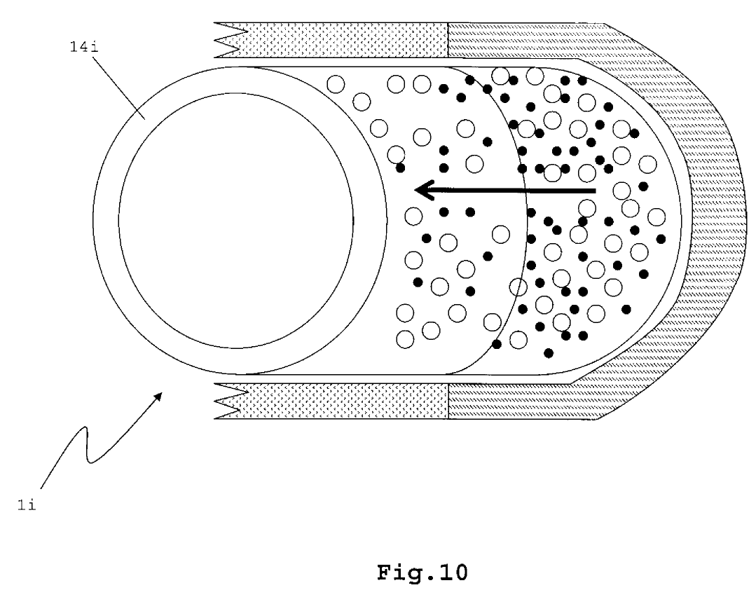

FIG. 10

FIG. 10 illustrates an implant 1i similar to that of FIG. 9, but the implant 1i has a hollow core.

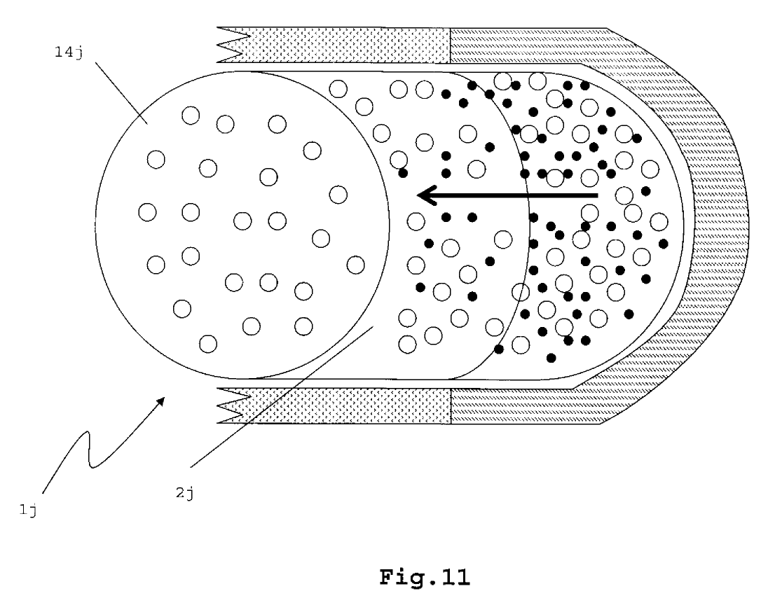

FIG. 11

In FIG. 11, an implant is similar to that of FIG. 9, but the solid implant 1j is porous not only on the external surface 2j, but also throughout the volume of the implant 14j.

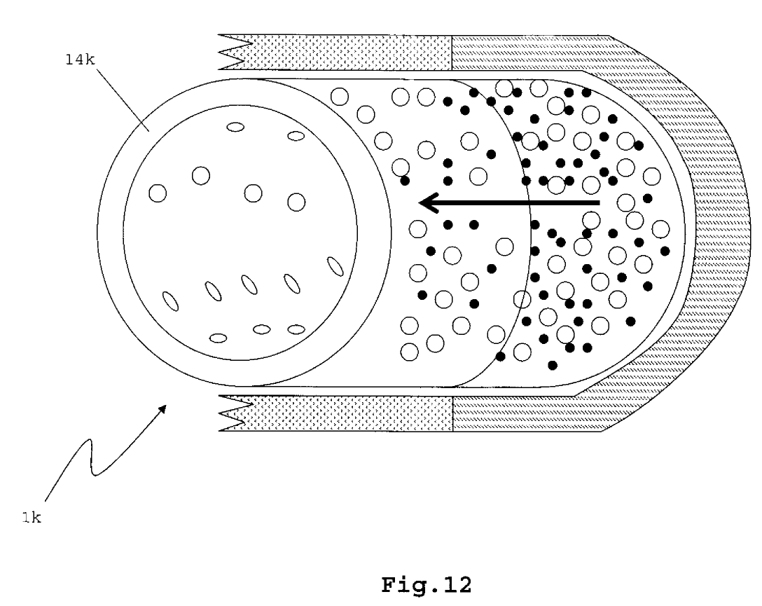

FIG. 12

In FIG. 12, the implant 1k is similar to that of FIG. 11, but the center of the implant 1k is hollow.

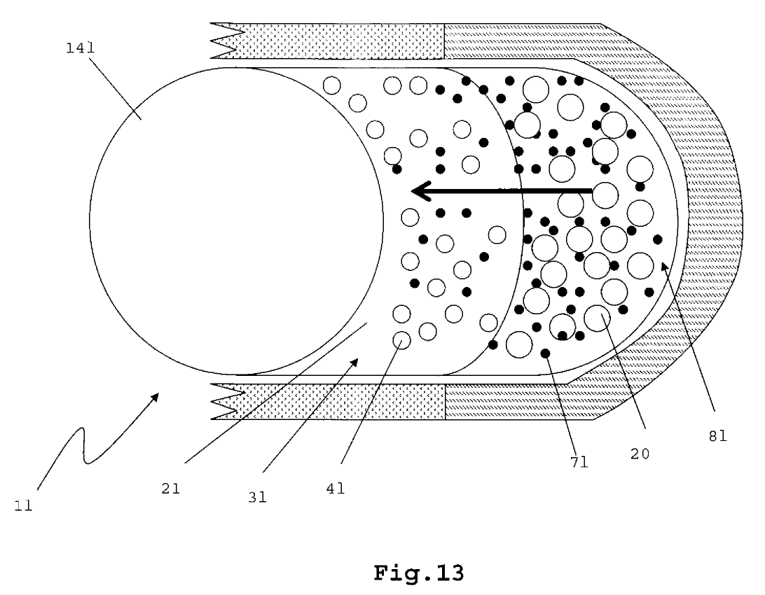

FIGS. 13-16

As shown in FIGS. 13-16, the implant 1l-1o may also have pores with sizes or densities that vary depending on which portion of the implant 1l-1o they are present. As shown in FIG. 13, an implant 1l with a solid interior has a first region 3l made from a first material, such as collagen, and a second region 8l made from a second material, such as ceramic. Pores 4l are relatively small over the external surface 2l within the first region, in which the concentration of the bioactive agent 7l is relatively low. Pores 20 over the external surface 2l in the second region are larger, and the concentration of the bioactive agent 7l is also higher. Alternatively, or additionally, the pores 20 may be in greater density within the second region 8l than the pores 4l in the first region 3l.

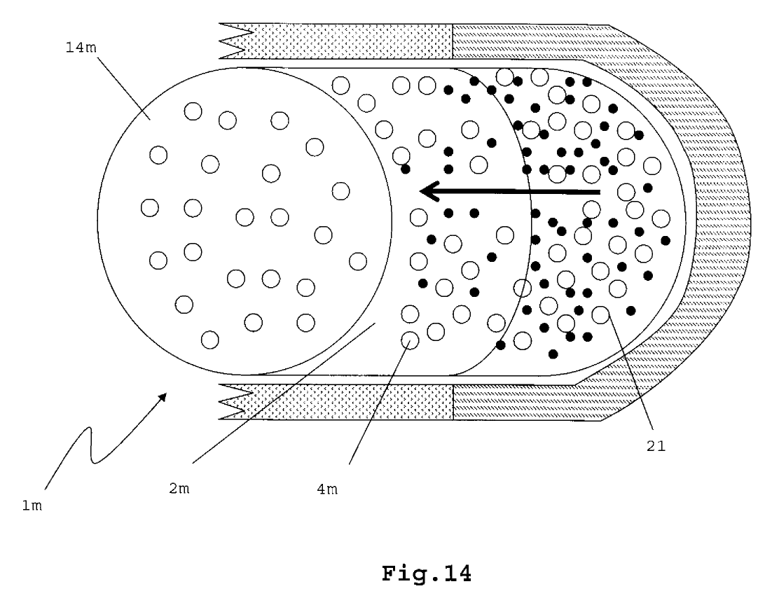

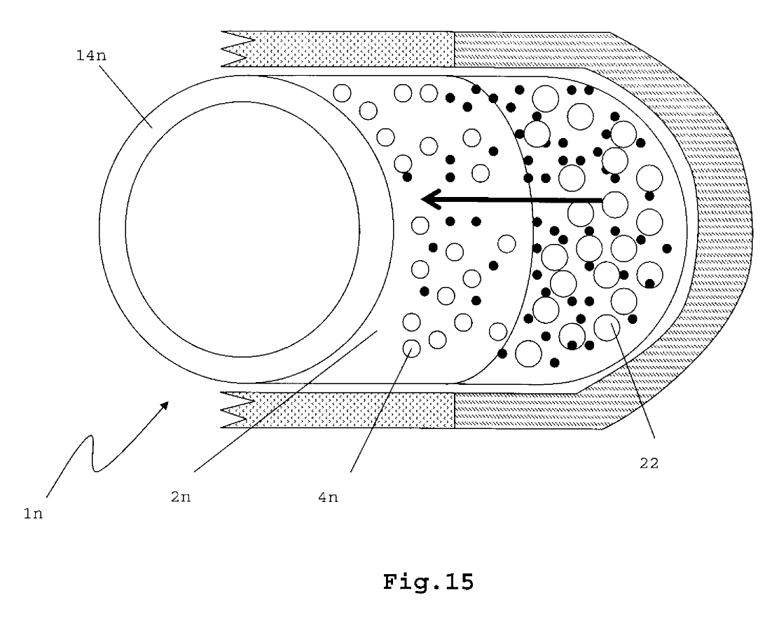

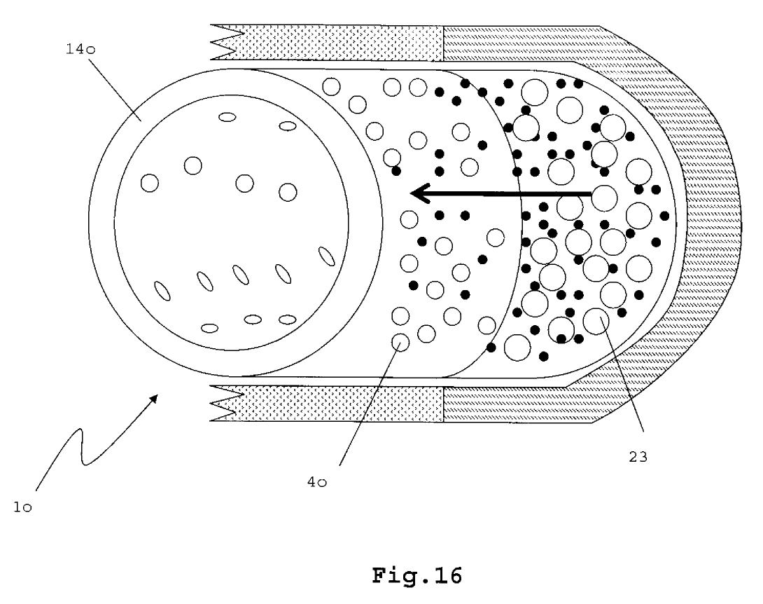

In FIG. 14, pores 4m, 21 are present not only on the external surface 2m of the implant 1m, but are also dispersed throughout the volume 14m of the implant 1m. In FIG. 15, an implant 1n has a hollow interior, and pores 4n, 22 are distributed across the external surface 2n of the implant 1n, wherein the pores are of various sizes. In FIG. 16, an implant 1o similar to that of FIG. 15 has pores 4o, 23 that are disposed throughout the volume of the tube-shaped body 14o.

In the above embodiments, different cocktails of bioactive agents may be included within the implant of the implant, each with its respective concentration gradient.

Additives Associated with the Implant

The porous structure of the implant may allow for optimal loading of its porous structure with bioactive agents in addition to growth factors or cells. The bioactive agent may be included within the pores of the implant, and/or may also be embedded within the implant material.

Suitable bioactive agents include, without limitation, growth factors (including osteogenic and chondrogenic agents), anti-inflammatory agents, pain-reducing agents, antibiotics, cells, and any combinations thereof.

Suitable bioactive agents include, without limitation, BMP-1, BMP-2, rhBMP-2, BMP-3, BMP-4, rhBMP-4, BMP-5, BMP-6, rhBMP-6, BMP-7[OP-1], rhBMP-7, BMP-8, BMP-9, BMP-10, BMP-11, BMP-12, BMP-13, BMP-14, BMP-15, BMP-16, BMP-17, BMP-18, Growth and Differentiation Factors, GDF-5, Cartilage Derived Morphogenic Proteins, LIM mineralization protein, platelet derived growth factor (PDGF), transforming growth factor β (TGF-β), insulin-related growth factor-I (IGF-I), insulin-related growth factor-II (IGF-II), fibroblast growth factor (FGF), beta-2-microglobulin (BDGF II), and rhGDF-5.

Suitable antibiotics include, without limitation nitroimidazole antibiotics, tetracyclines, penicillins, cephalosporins, carbopenems, aminoglycosides, macrolide antibiotics, lincosamide antibiotics, 4-quinolones, rifamycins and nitrofurantoin. Suitable specific compounds include, without limitation, ampicillin, amoxicillin, benzylpenicillin, phenoxymethylpenicillin, bacampicillin, pivampicillin, carbenicillin, cloxacillin, cyclacillin, dicloxacillin, methicillin, oxacillin, piperacillin, ticarcillin, flucloxacillin, cefuroxime, cefetamet, cefetrame, cefixine, cefoxitin, ceftazidime, ceftizoxime, latamoxef, cefoperazone, ceftriaxone, cefsulodin, cefotaxime, cephalexin, cefaclor, cefadroxil, cefalothin, cefazolin, cefpodoxime, ceftibuten, aztreonam, tigemonam, erythromycin, dirithromycin, roxithromycin, azithromycin, clarithromycin, clindamycin, paldimycin, lincomycirl, vancomycin, spectinomycin, tobramycin, paromomycin, metronidazole, tinidazole, ornidazole, amifloxacin, cinoxacin, ciprofloxacin, difloxacin, enoxacin, fleroxacin, norfloxacin, ofloxacin, temafloxacin, doxycycline, minocycline, tetracycline, chlortetracycline, oxytetracycline, methacycline, rolitetracyclin, nitrofurantoin, nalidixic acid, gentamicin, rifampicin, amikacin, netilmicin, imipenem, cilastatin, chloramphenicol, furazolidone, nifuroxazide, sulfadiazin, sulfametoxazol, bismuth subsalicylate, colloidal bismuth subcitrate, gramicidin, mecillinam, cloxiquine, chlorhexidine, dichlorobenzylalcohol, methyl-2-pentylphenol or any combination thereof.

Suitable anti-inflammatory compounds include the compounds of both steroidal and non-steroidal structures.

Suitable non-limiting examples of steroidal anti-inflammatory compounds are corticosteroids such as hydrocortisone, cortisol, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluocinolone, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone. Mixtures of the above steroidal anti-inflammatory compounds can also be used.

Non-limiting example of non-steroidal anti-inflammatory compounds include nabumetone, celecoxib, etodolac, nimesulide, apasone, gold, oxicams, such as piroxicam, isoxicam, meloxicam, tenoxicam, sudoxicam, and CP-14,304; the salicylates, such as aspirin, disalcid, benorylate, trilisate, safapryn, solprin, diflunisal, and fendosal; the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; the fenamates, such as mefenamic, meclofenamic, flufenamic, niflumic, and tolfenamic acids; the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and the pyrazoles, such as phenylbutazone, oxyphenbutazone, feprazone, azapropazone, and trimethazone.

The various compounds encompassed by this group are well-known to those skilled in the art. For detailed disclosure of the chemical structure, synthesis, side effects, etc. of non-steroidal anti-inflammatory compounds, reference may be had to standard texts, including Anti-inflammatory and Anti-Rheumatic Drugs, K. D. Rainsford, Vol. I-III, CRC Press, Boca Raton, (1985), and Anti-inflammatory Agents, Chemistry and Pharmacology 1, R. A. Scherrer, et al., Academic Press, New York (1974), each incorporated herein by reference.

Mixtures of these non-steroidal anti-inflammatory compounds may also be employed, as well as the pharmologically acceptable salts and esters of these compounds.

In addition, so-called “natural” anti-inflammatory compounds are useful in methods of the disclosed invention. Such compounds may suitably be obtained as an extract by suitable physical and/or chemical isolation from natural sources (e.g., plants, fungi, by-products of microorganisms).

Suitable non-limiting examples of such compounds include candelilla wax, alpha bisabolol, aloe vera, Manjistha (extracted from plants in the genus Rubia, particularly Rubia Cordifolia), and Guggal (extracted from plants in the genus Commiphora, particularly Commiphora Mukul), kola extract, chamomile, sea whip extract, compounds of the Licorice (the plant genus/species Glycyrrhiza glabra) family, including glycyrrhetic acid, glycyrrhizic acid, and derivatives thereof (e.g., salts and esters).

Suitable salts of the foregoing compounds include metal and ammonium salts. Suitable esters include C₂-C₂₄ saturated or unsaturated esters of the acids, preferably C₁₀-C₂₄, more preferably C₁₆-C₂₄. Specific examples of the foregoing include oil soluble licorice extract, the glycyrrhizic and glycyrrhetic acids themselves, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, and disodium 3-succinyloxy-beta-glycyrrhetinate.

Generally, anti-inflammatory non-steroid drugs are included in the definition of pain-reducing agents because they provide pain relief. In addition, suitable pain-reducing agents include other types of compounds, such as, for example, opioids (such as, for example, morphine and naloxone), local anaesthetics (such as, for example, lidocaine), glutamate receptor antagonists, α-adrenoreceptor agonists, adenosine, canabinoids, cholinergic and GABA receptors agonists, and different neuropeptides. A detailed discussion of different analgesics is provided in Sawynok et al., (2003) Pharmacological Reviews, 55:1-20, the content of which is incorporated herein by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

What is claimed is:

1. An implantable device comprising an implant having a concentration gradient of a growth factor.

2. The device of claim 1 wherein the implant has a hollow interior.

3. The device of claim 1 wherein the implant has a solid interior.

4. The device of claim 1 wherein the implant has exterior surfaces that are porous and interior surfaces that are non-porous.

5. The device of claim 1 wherein the implant has exterior surfaces and interior surfaces that are porous.

6. The device of claim 1 wherein the implant has a shape that is cylindrical, oval, square, cubic, sheet-like, star shaped, or plug-like.

7. The device of claim 1 wherein the implant is made from one or more materials selected from the group consisting of synthetic biodegradable polymers, synthetic non-biodegradable polymers, natural polymers, ceramics and combinations thereof.

8. The device of claim 7, wherein the ceramics are selected from the group consisting of hydroxyapatite (HA), tri-calcium phosphate, and combinations thereof.

9. The device of claim 7 wherein the implant is made from synthetic degradable polymers selected from the group consisting of polylactic acid, polyglycolic acid, polylactic-polyglycolic acid, polyorthoesters, Polycapralactone, Polydiaxonone, Polyvynal Fumarate, and combinations thereof.

10. The device of claim 7, wherein the implant is made from synthetic non-degradable polymers selected from the group consisting of delrin, polyurethane, polyethylene, hydrogels, and combinations thereof.

11. The device of claim 7 wherein the implant is made from natural polymers selected from the group consisting of collagen, elastin, silk, hyaluronic acid, chytosan, and combinations thereof.

12. The device of claim 1 wherein the implant is capable of insertion into a surgically prepared defect to promote growth of dissimilar tissue.

13. The device of claim 12 wherein the dissimilar tissue is bone and cartilage tissue.

14. The device of claim 13 wherein a first region of the implant is adapted for contact with the cartilage tissue and has a lower concentration of the growth factor than a second region of the implant that is adapted for contacting the bone tissue.

15. The device of claim 14 wherein the growth factor has a concentration in the range of 0.05 to 0.5 mg/mL in the first region.

16. The device of claim 14 wherein the growth factor has a concentration in the range of 0.5 to 4 mg/mL in the second region.

17. The device of claim 14 wherein the second region is made from a compound comprising calcium phosphate.

18. The device of claim 14 wherein the first region is made from a natural polymer.

19. The device of claim 18 wherein the nature polymer is collagen or chitosan.

20. The device of claim 12 wherein the implant has a first region with a lower concentration of growth factors than a second region of the implant.

21. The device of claim 20 further comprising a plurality of pores, wherein pores associated with the second region have larger average diameters than pores associated with the first region.

22. The invention of claim 20 further comprising a plurality of pores, wherein the pores associated with the second region have an average diameter substantially equal to an average diameter of the pores associated with the first region.

23. The device of claim 1 wherein the growth factor is selected from the group consisting of BMP-1, BMP-2, rhBMP-2, BMP-3, BMP-4, rhBMP-4, BMP-5, BMP-6, rhBMP-6, BMP-7[OP-1], rhBMP-7, BMP-8, BMP-9, BMP-10, BMP-11, BMP-12, BMP-13, BMP-14, BMP-15, BMP-16, BMP-17, BMP-18, Growth and Differentiation Factors, GDF-5, Cartilage Derived Morphogenic Proteins, LIM mineralization protein, platelet derived growth factor (PDGF), transforming growth factor α (TGF-α), insulin-related growth factor-I (IGF-I), insulin-related growth factor-II (IGF-II), fibroblast growth factor (FGF), beta-2-microglobulin (BDGF II), and rhGDF-5, and combinations thereof.
